(19) United States
(12) Patent Application Publication
(10) Pub. No.: US 2024/0100225 A1
(43) Pub. Date: Mar. 28, 2024

(54) METHOD OF MANUFACTURING ARTIFICIAL SKIN STRUCTURE APPLIED WITH HAPTIC TECHNOLOGY AND ARTIFICIAL SKIN STRUCTURE MANUFACTURED BY THE SAME

(71) Applicant: Korea University Of Technology And Education Industry-University Cooperation Foundation, Cheonan-si (KR)

(72) Inventors: Sang-Youn KIM, Seoul (KR); Seok-Han LEE, Cheonan-si (KR)

(73) Assignee: Korea University Of Technology And Education Industry-University Cooperation Foundation, Cheonan-si (KR)

(21) Appl. No.: 18/304,227

(22) Filed: Apr. 20, 2023

(30) Foreign Application Priority Data
Sep. 27, 2022 (KR) 10-2022-0122499

(51) Int. Cl. A61L 27/52; A61L 27/16; A61L 27/60; G06F 3/044

(52) U.S. Cl. CPC A61L 27/52; A61L 27/16; A61L 27/60; G06F 3/0447; G06F 2203/04102; G06F 2203/04103

(57) ABSTRACT

Disclosed is a method of manufacturing an artificial skin structure applied with a haptic technology, in which a polymer layer made of a PVC gel material is interposed between electrodes made of a hydrogel material to accurately recognize a pressing form, a pressing direction, and a pressing degree of external force based on a capacitance change value or resistance change value to the external force applied to the electrode made of a hydrogel material, and particularly, a material having excellent elasticity is applied to an electrode and a polymer layer itself to accurately recognize and utilize various motions, such as stroking, pinching, and twisting motions, instead of a simple pressing motion, and an artificial skin structure manufactured by the same.

### CROSS-REFERENCE TO RELATED APPLICATIONS

This application claims priority to and the benefit of Korean Patent Application No. 10-2022-0122499 filed in the Korean Intellectual Property Office on Sep. 27, 2022, the entire contents of which are incorporated herein by reference.

### TECHNICAL FIELD

The present invention relates to a method of manufacturing an artificial skin structure applied with haptic technology and an artificial skin structure manufactured by the same, and more particularly, to a method of manufacturing an artificial skin structure applied with a haptic technology, in which a polymer layer made of a PVC gel material is interposed between electrodes made of a hydrogel material to accurately recognize a pressing form, a pressing direction, and a pressing degree of external force based on a capacitance change value or resistance change value to the external force applied to the electrode made of a hydrogel material, and particularly, a material having excellent elasticity is applied to an electrode and a polymer layer itself to accurately recognize and utilize various motions, such as stroking, pinching, and twisting motions, instead of a simple pressing motion, and an artificial skin structure manufactured by the same.

### BACKGROUND ART

In general, robots are utilized in a variety of fields, including medical and industrial fields. Recently, robots, such as a humanoid, that have a form similar to a human body and mimic human behavior have been developed. Humanoid robots mimic human intelligence, behavior, senses, interactions, and the like and aim to take over human tasks or cooperate with humans for various interactions and exchanges.

Such robots may be equipped with artificial skin (for example, E-skin) that resembles human skin, and in most artificial skin, a protective film with low oxygen permeability is provided on a polymeric substrate to detect the tactile sensation of a user's touch, and the detected tactile sensation is utilized for a control of the motion of the robot as a control signal.

However, in the case of artificial skin developed in the related art, there is a limitation in that the artificial skin can only be implemented in a partial area because the artificial skin is made in the form of a patch having a narrow area and thus it cannot be applied to a large area. In addition, due to the material characteristics, it is difficult to apply the artificial skin to areas with movement such as bends and joints, so various problems, such as restrictions on the movement of the robot to which the artificial skin is applied, occur.

### PRIOR ART LITERATURE

### Patent Document

Korean Patent Application Laid-Open No. 10-2022-0073128

### SUMMARY OF THE INVENTION

The present invention is conceived in response to the background art, and has been made in an effort to provide a method of manufacturing an artificial skin structure applied with a haptic technology, in which a polymer layer made of a PVC gel material is interposed between electrodes made of a hydrogel material to accurately recognize a pressing form, a pressing direction, and a pressing degree of external force based on a capacitance change value or resistance change value to the external force applied to the electrode made of a hydrogel material, and particularly, a material having excellent elasticity is applied to an electrode and a polymer layer itself to accurately recognize and utilize various motions, such as stroking, pinching, and twisting motions, instead of a simple pressing motion, and an artificial skin structure manufactured by the same.

An exemplary embodiment of the present invention provides an artificial skin structure applied with haptic technology, the artificial skin structure including: an upper electrode 110 having elasticity; a lower electrode 120 positioned under the upper electrode 110 and having elasticity; and a polymer layer 130 interposed between the upper electrode 110 and the lower electrode 120, in which wherein the artificial skin structure is configured to recognize a pressing form, a pressing direction, and a pressing degree of external force applied to the upper electrode 110.

In the exemplary embodiment, the upper electrode 110 and the lower electrode 120 may be made of a transparent hydrogel material, and be manufactured to have elasticity in a side-to-side direction.

In the exemplary embodiment, a transparent PVC gel material may be applied to the polymer layer 130.

In the exemplary embodiment, each of the upper electrode 110 and the lower electrode 120 may be electrically connected with an electric wire for conducting current.

In the exemplary embodiment, the pressing form, the pressing direction, and the pressing degree of the external force may be recognized based on a capacitance change value between the upper electrode 110 and the lower electrode 120 due to external force applied to the upper electrode 110.

In the exemplary embodiment, the pressing form, the pressing direction, and the pressing degree of the external force may be recognized based on a resistance change value between the upper electrode 110 and the lower electrode 120 due to external force applied to the upper electrode 110.

In the exemplary embodiment, a plurality of upper electrodes 110 and a plurality of lower electrodes 120 may be arranged with the polymer layer 130 having a predetermined area interposed therebetween, and a pressing form, a pressing direction, and a pressing degree of external force that is collected from other upper electrodes 110 adjacent to the specific upper electrode 110 to which the external force is applied may be recognized.

In the exemplary embodiment, a transparent PVC gel material may be applied to the polymer layer 130, and a transparent hydrogel material may be applied to the plurality of upper electrodes 110 and the plurality of lower electrodes 120.

Another exemplary embodiment of the present invention provides a method of manufacturing an artificial skin structure applied with haptic technology, the method including: washing the polymer layer made of a polyvinyl chloride gel material with methanol; applying a surface treatment solution to the polymer layer to perform a surface treatment; washing an application surface on which the surface treatment solution has been applied with methanol at least three times, drying the application surface with nitrogen gas, and attaching a mask for forming a structure; filling a hydrogel solution into grooves formed in the mask for forming the structure; and irradiating the hydrogel solution with ultraviolet light in a UV chamber to chemically covalently bond the hydrogel solution to the polymer layer according to the shape of the grooves.

According to the exemplary embodiments of the present invention, the present invention has the advantage in that it is possible to accurately recognize the pressing form, the pressing direction, and the pressing degree of external force applied to an electrode of a hydrogel material based on a capacitance change value or a resistance change value.

In particular, the present invention has the advantage in that a material having excellent elasticity is applied to an electrode and a polymer layer itself, so that it is possible to accurately recognize and utilize various motions, such as stroking, pinching, and twisting motions, instead of a simple pressing motion.

### BRIEF DESCRIPTION OF THE DRAWINGS

FIG. 1 is a diagram illustrating an artificial skin structure 100 applied with haptic technology and a shape of a single cell formed by the artificial skin structure 100 according to an exemplary embodiment of the present invention.

FIG. 2 is an exploded perspective view of the artificial skin structure 100 applied with haptic technology illustrated in FIG. 1.

FIG. 3 is a diagram illustrating an array of a plurality of artificial skin structures 100 applied with haptic technology illustrated in FIG. 1.

FIG. 4 is a diagram illustrating a concept of recognizing a pressing form, a pressing direction, and a pressing degree of external force based on a capacitance change value or a resistance change value between an upper electrode 110 and a lower electrode 120.

FIG. 5 is a flowchart illustrating a method of manufacturing an artificial skin structure applied with haptic technology illustrated in FIG. 1 in a sequential order.

### DETAILED DESCRIPTION

Hereinafter, exemplary embodiments are presented to facilitate an understanding of the present invention. However, the following exemplary embodiments are provided to facilitate understanding of the invention and are not intended to limit the scope of the invention by the exemplary embodiment.

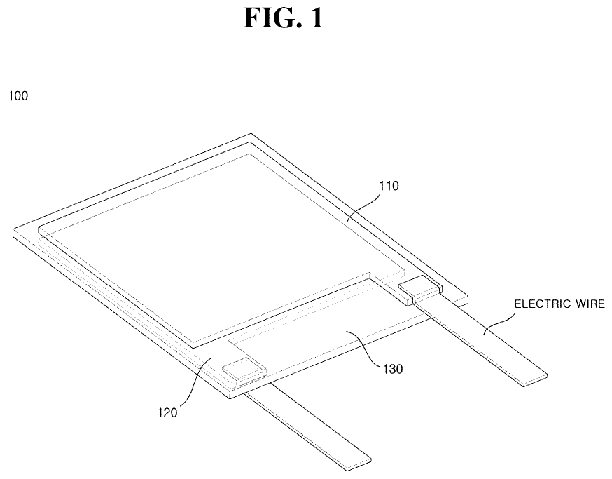

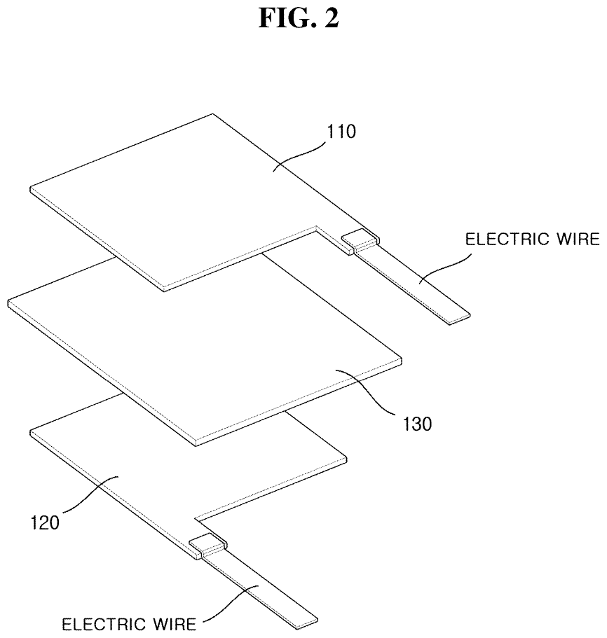

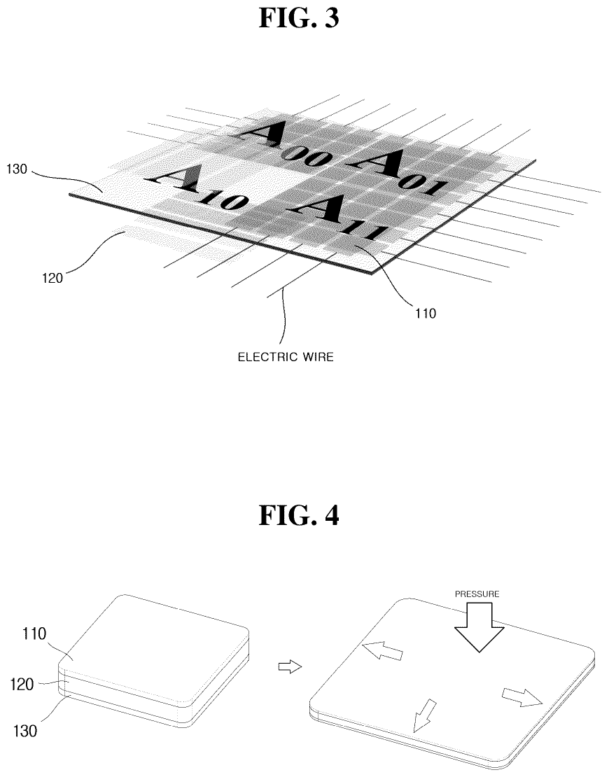

FIG. 1 is a diagram illustrating an artificial skin structure 100 applied with haptic technology and a shape of a single cell formed by the artificial skin structure 100 according to an exemplary embodiment of the present invention, FIG. 2 is an exploded perspective view of the artificial skin structure 100 applied with haptic technology illustrated in FIG. 1, and FIG. 3 is a diagram illustrating an array of a plurality of artificial skin structures 100 applied with haptic technology illustrated in FIG. 1.

Referring to FIGS. 1 to 3 together, an artificial skin structure 100 applied with haptic technology according to an exemplary embodiment of the present invention may include an upper electrode 110, a lower electrode 120, and a polymer layer 130.

First, the upper electrode 110 and the lower electrode 120 may each be made of a hydrogel material that is elastic in the side-to-side direction but is transparent.

The polymer layer 130 is interposed between the upper electrode 110 and the lower electrode 120.

The polymer layer 130 may also be made of a PVC gel material that is elastic in the side-to-side direction but is transparent.

Thus, since the upper electrode 110, the lower electrode 120, and the polymer layer 130 are all elastic in the side-to-side direction, the motion of pressing, rubbing, pulling, stroking, pinching, and twisting the upper electrode 110 may be mimicked similarly to real skin. This means a structure in which the upper electrode 110 and the lower electrode 120 corresponding to the stretchable electrodes are patterned on a dielectric polymer that is the polymer layer 130.

Since each of the upper electrode 110 and the lower electrode 120 may be electrically connected with an electrical wire for conducting current and each of the upper electrode 110 and the lower electrode 120 has a different electrode, external force applied to the upper electrode 110 causes an electrical stimulus to the lower electrode 120, which induces the generation of an electrical signal.

The upper electrode 110, the lower electrode 120, and the polymer layer 130 may have a modularized structure as a single cell, and a plurality of upper electrodes 110 and a plurality of lower electrodes 120 may be arranged and patterned with the polymer layers 130 having a predetermined area (the size of the predetermined area is not limited herein) interposed therebetween, as illustrated in FIG. 2.

In this case, other upper electrodes 110 adjacent to the specific upper electrode 110 to which the external force is applied may be physically stimulated, such as being pulled or pushed, so that the pressing form, the pressing direction, and the pressing degree of the external force collected from other upper electrodes 110 may be recognized.

On the other hand, the pressing form, the pressing direction, and the pressing degree of the external force may be recognized based on the capacitance change value or the resistance change value between the upper electrode 110 and the lower electrode 120, which will be described below.

FIG. 4 is a diagram illustrating a concept of recognizing the pressing form, the pressing direction, and the pressing degree of external force based on the capacitance change value or the resistance change value between the upper electrode 110 and the lower electrode 120.

Referring to FIG. 4, the polymer layer 130 is interposed between the upper electrode 110 and the lower electrode 120, and in this case, the upper electrode 110 and the lower electrode 120 correspond to hydrogel materials and the polymer layer 130 corresponds to a polyvinyl chloride gel material.

When external force is applied to the top surface of the upper electrode 110, the total area of the upper electrode 110, the lower electrode 120, and the polymer layer 130 becomes larger, but the gap between the upper electrode 110 and the lower electrode 120 becomes narrower, and the capacitance increases accordingly, which induces a change in the capacitance. This may be expressed as follows.

$$C = {ε}_{0} {ε}_{r} \frac{A}{d}$$

C: Capacitance
ε₀: Permittivity of vacuum

(ε₀≈8.854×10⁻¹² F·m⁻¹)
εᵣ: Relative permittivity/dielectric constant of a dielectric located between electrodes
A: Width of electrode plate
D: Distance between electrode plates

While the above example is based on the case where the artificial skin structure 100 applied with haptic technology is a single cell, the present invention may be applied identically to the case of the arrayed structure illustrated in FIG. 3 discussed above.

When external force, such as top-to-bottom pressing force, is applied to a specific single cell, stroking force, or pinching or twisting force, external force corresponding to the above applied external force is applied to other single cells adjacent to the corresponding single cell. For example, the external force, such as pinching force or twisting force, may apply stimulation that other single cells are pulled in the corresponding direction, twisted together, and the like, so that a change in capacitance may occur in all single cells associated with the other single cells. Thus, in the present invention, based on the capacitance change value, it is possible to recognize the pressing form, the pressing direction, and the pressing degree of the external force.

In another example, the polymer layer 130 is interposed between the upper electrode 110 and the lower electrode 120, and in this case, the upper electrode 110 and the lower electrode 120 correspond to a hydrogel material and the polymer layer 130 corresponds to a polyvinyl chloride gel material.

When external force is applied to the upper surface of the upper electrode 110, the thickness of the polymer layer 130 interposed between the upper electrode 110 and the lower electrode 120 becomes thinner, but the areas of the upper electrode 110 and the lower electrode 120 becomes larger, and the resistance is lowered accordingly, thereby inducing a resistance change. This may be expressed as follows.

$$R = ρ \frac{L}{A}$$

R: Resistance
p: Specific resistance of polyvinyl chloride gel
L: Thickness of polyvinyl chloride gel located between hydrogel electrodes
A: Width of hydrogel electrode

The above example may likewise be applied identically to the case of the arrayed structure of FIG. 3 discussed above.

When external force, such as top-to-bottom pressing force, is applied to a specific single cell, stroking force, or pinching or twisting force, external force corresponding to the above applied external force is applied to other single cells adjacent to the corresponding single cell. For example, the external force, such as pinching force or twisting force, may apply stimulation that other single cells are pulled in the corresponding direction, twisted together, and the like, so that a change in resistance may occur in all single cells associated with the other single cells. Thus, in the present invention, based on the resistance change value, it is possible to recognize the pressing form, the pressing direction, and the pressing degree of the external force.

Next, the method of manufacturing the artificial skin structure 100 applied with the haptic technology described above will be discussed.

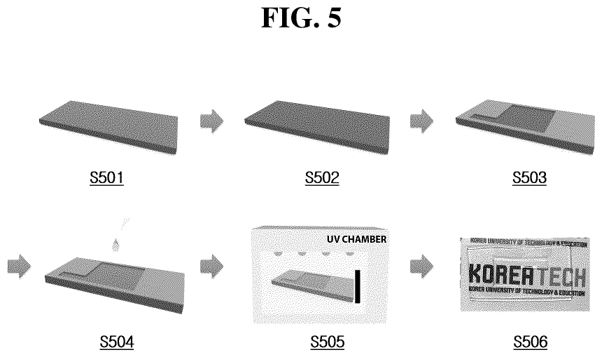

FIG. 5 is a flowchart illustrating a method of manufacturing an artificial skin structure applied with haptic technology illustrated in FIG. 1 in a sequential order.

Referring to FIG. 5, a polymer layer made of a polyvinyl chloride gel (PAAm pre-gel) material is first washed with methanol (S501), and then a surface treatment is performed by applying a surface treatment solution to the polymer layer (S502). In this case, the surface treatment solution is a solution prepared by adding ethanol to 10 weight percent (%) of benzophenone. The surface treatment process lasts two minutes.

Next, the application surface to which the surface treatment solution has been applied is washed at least three times with methanol, the application surface is dried with nitrogen gas, a mask for forming a structure is attached (S503), and a hydrogel solution is filled into the grooves formed in the mask for forming the structure (S504). Here, the grooves formed in the mask for forming the structure become the shapes of the previously discussed upper electrode 110 and lower electrode 120.

Next, the hydrogel solution is irradiated with ultraviolet light in a UV chamber to chemically covalently bond the hydrogel solution to the polyvinyl chloride gel polymer layer according to the shape of the grooves (S505). In this case, the UV light may be set to a wavelength of 365 nm and the UV irradiation may be performed for one hour. Thereafter, the polyvinyl chloride gel and the hydrogel are chemically bonded to form a shape (S506).

In the forgoing, the present invention has been described with reference to the exemplary embodiment of the present invention, but those skilled in the art may appreciate that the present invention may be variously corrected and changed within the range without departing from the spirit and the area of the present invention described in the appending claims.

What is claimed is:

1. An artificial skin structure applied with haptic technology, the artificial skin structure comprising:
an upper electrode having elasticity;
a lower electrode positioned under the upper electrode and having elasticity; and
a polymer layer interposed between the upper electrode and the lower electrode,
wherein the artificial skin structure is configured to recognize a pressing form, a pressing direction, and a pressing degree of external force applied to the upper electrode.

2. The artificial skin structure of claim 1, wherein the upper electrode and the lower electrode are made of a transparent hydrogel material, and are manufactured to have elasticity in a side-to-side direction.

3. The artificial skin structure of claim 1, wherein a transparent PVC gel material is applied to the polymer layer.

4. The artificial skin structure of claim 1, wherein each of the upper electrode and the lower electrode is electrically connected with an electric wire for conducting current.

5. The artificial skin structure of claim 1, wherein the pressing form, the pressing direction, and the pressing degree of the external force are recognized based on a capacitance change value between the upper electrode and the lower electrode due to external force applied to the upper electrode.

6. The artificial skin structure of claim 1, wherein the pressing form, the pressing direction, and the pressing degree of the external force are recognized based on a resistance change value between the upper electrode and the lower electrode due to external force applied to the upper electrode.

7. The artificial skin structure of claim 1, wherein a plurality of upper electrodes and a plurality of lower electrodes are arranged with the polymer layer having a predetermined area interposed therebetween, and
a pressing form, a pressing direction, and a pressing degree of external force that is collected from other upper electrodes adjacent to the specific upper electrode to which the external force is applied are recognized.

8. The artificial skin structure of claim 7,
wherein a transparent PVC gel material is applied to the polymer layer, and
a transparent hydrogel material is applied to the plurality of upper electrodes and the plurality of lower electrodes.

9. A method of manufacturing an artificial skin structure applied with haptic technology, the method comprising:
washing the polymer layer made of a polyvinyl chloride gel material with methanol;
applying a surface treatment solution to the polymer layer to perform a surface treatment;
washing an application surface on which the surface treatment solution has been applied with methanol at least three times, drying the application surface with nitrogen gas, and attaching a mask for forming a structure;
filling a hydrogel solution into grooves formed in the mask for forming the structure; and
irradiating the hydrogel solution with ultraviolet light in a UV chamber to chemically covalently bond the hydrogel solution to the polymer layer according to the shape of the grooves.
